(19) United States
(12) Patent Application Publication
(10) Pub. No.: US 2007/0178155 A1
(43) Pub. Date: Aug. 2, 2007

(54) Preparation for gastric buoyant sustained drug release dosage form

(76) Inventor: David YiHai Jiang, Irvine, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/345,608

(22) Filed: Jan. 31, 2006

(51) Int. Cl. A61K 38/13; A61K 31/43; A61K 31/545; A61K 31/519; A61K 9/46; A61K 35/20

(52) U.S. Cl. 424/466; 424/535; 514/11; 514/192; 514/200; 514/355; 514/166; 514/423; 514/262.1

(57) ABSTRACT

Disclosed is a floating sustained release pharmaceutical dosage form including a drug that is adapted to release the drug over an extended period of time. The buoyant pharmaceutical dosage form provides extended gastric residence time of the formulation so that substantially all of the drug is released in the stomach over an extended period. The pharmaceutical dosage form is formulated with low molecular weight concentrated milk proteins to provide buoyancy to the dosage form which can float in gastric fluid for an extended period, including up to about 48 hours.

### BACKGROUND OF THE INVENTION

1. Field of the Invention

The present invention relates to extended or sustained release pharmaceutical formulations. More specifically, it relates to formulations which are buoyant in gastric fluid and thereby have increased gastric residence time in which to deliver one or more drugs.

2. Description of the Related Art

In some circumstances, it is desirable to administer a single oral dose of a selected drug during the treatment of a patient where the drug is released over an extended of period of time, rather than administering several doses at regular intervals. Many sustained release formulations are known in the pharmaceutical arts, such formulations being intended to continuously provide a drug for absorption while the dosage form passes through the gastrointestinal tract of the patient. The conventional oral dosage form generally releases the active compound in a period of anywhere from a few minutes to two hours after administration, making repeated administration of the dosage form necessary. Thus, a sustained release formulation which releases the drug over an extended period of time can be advantageous. However, conventional sustained released formulations which are not retained in the stomach and therefore release the drug, at least partially, in the intestine are not suitable for medicaments which are principally absorbed from the stomach, such as acidic medicament, and antacids.

### SUMMARY OF THE INVENTION

The present invention is directed to a sustained release pharmaceutical dosage form that is adapted to release a drug over an extended period of time. The dosage form preferably provides extended gastric residence time for the formulation so that substantially all of the drug is released in the stomach over an extended period. The described pharmaceutical dosage form is formulated to be buoyant in the stomach, the floating dosage form including low molecular weight concentrated milk proteins, optional fatty material, and optional polymers which have the properties of a gelling agent and which upon contact with gastric fluid hydrate and form a gelatinous barrier or mass. The pharmaceutical dosage form is preferably buoyant in gastric fluid for a period of up to about 48 hours.

The stomach-directed drug delivery system of preferred embodiments is conceptually designed to optimize gastric delivery of antacid, anti-viral, anti-hypertension, anti-diabetes, and anti-microbial compositions and any acid-stable medications. In preferred embodiments, the disclosed floating dosage form provides greater flexibility for drug release profile adjustment and/or can be directly compressed without granulation. In certain embodiments, the drug release profile of the buoyancy formulations can achieve zero order release.

In accordance with one preferred embodiment, there is provided a pharmacologically active gastric buoyant composition having extended gastric residence time with sustained drug release. The composition comprises at least one pharmacologically active compound, at least one pharmacologically acceptable additive, low molecular weight concentrated milk proteins, optionally one or more fatty materials, and optionally one or more polymers. The one or more polymers includes swellable and non-swellable polymers and hydrophilic and hydrophobic polymers. The polymer material preferably has a number average molecular weight of between about 1,000 and 20,000,000 grams per mole. In preferred embodiments, the low molecular weight concentrated milk proteins and polymer(s) are present in the form of a substantially homogeneous mixture and/or the weight ratio low molecular weight concentrated milk proteins to polymer(s) ranges from 20:80 to 100:0, including about 5:1 to about 15:1 and/or the composition is in dry compressed state. In a preferred embodiment, the composition is compressed to a hardness of less than about 25 kp, including about 10-14 kp.

### BRIEF DESCRIPTION OF THE DRAWINGS

FIG. 1 illustrates a drug release curve for a formulation according to Example 1.

FIG. 2 illustrates a drug release curve for a formulation according to Example 2.

FIG. 3 illustrates a drug release curve for a formulation according to Example 3.

FIG. 4 illustrates a drug release curve for a formulation according to Example 4.

FIG. 5 illustrates a drug release curve for a formulation according to Example 5.

### DETAILED DESCRIPTION OF THE PREFERRED EMBODIMENT

Formulations suitable for the preparation of sustained release tablets for oral administration are provided. The formulations comprise one or more medicaments in combination with low molecular weight concentrated milk proteins, optionally mixed with fatty materials and/or polymers so that, when placed in contact with gastric fluid, the formulation is buoyant and thus is retained in the stomach during a time when substantially all of the one or more medicaments are released therefrom for a period from at least about 8 hours, at least about 10 hours, at least about 12 hours, at least about 15 hours, at least about 18 hours, at least about 20 hours, at least about 24 hours, or at least about 30 hours, up to about 48 hours.

Preferred embodiments comprise a sustained release buoyant pharmaceutical dosage form, which includes a drug, wherein the form is adapted to release the drug over an extended period of time, preferably such that all or substantially all of the drug release takes place within the stomach. The pharmaceutical dosage form comprises proteins, preferably low molecular weight concentrated milk proteins, and optionally includes one or more fatty materials, and/or one or more polymers to provide in a formulation which is buoyant or becomes buoyant when exposed to gastric fluid. Although the dosage form can be utilized with any medicament or drug it is particularly useful for acid-stable medicaments and other drugs which are most advantageously released in the stomach. Furthermore, the terms “drug” and “medicament” as used herein are broad terms used in accordance with their ordinary meanings and include, but are not limited to, prescription pharmaceutical drugs, over-the-counter pharmaceuticals, nutritional supplements, nutraceuticals, herbs, herbal extracts, vitamins, minerals, amino acids, small peptides, biologicals such as recombinant proteins and other materials and other chemical elements, salts and compounds that are administered to a mammal for the treatment or prevention of a disease or condition, or to enhance the health or well-being of the mammal.

In accordance with preferred embodiments, formulations for sustained release tablets and other forms for oral administration are provided, which forms will remain floating in gastric fluid for an extended period of time. The term “dosage form” or “form” refers to the composition or formulation generally as it is administered to a patient or subject. Examples of dosage forms include, but are not limited to, tablets (including caplets), capsules, granules, pellets, and the like. In a preferred embodiment, the sustained release formulations comprise a generally homogeneous mixture of one or more medicaments with low molecular weight concentrated milk proteins. In some embodiments, the formulation optionally includes one or more fatty materials and/or one or more polymers. When the dosage form, such as a tablet, comes into contact with gastric fluid at body temperature, it forms a soft gelatinous mass on the surface of the tablet, causing it to float in the gastric fluid. The medicament is slowly released from the gelatinous mass which, due to its buoyancy, remains buoyant in the gastric fluid.

Upon oral ingestion of sustained release compositions, any film coating on the dosage form, if present, dissolves leaving the contents in contact with gastric fluid. Upon contact with gastric fluid, the outermost low molecular weight concentrated milk proteins and polymers (if present) of the tablet or other form hydrate to form an outside layer or barrier which enlarges the form somewhat, preferably while retaining the general shape thereof and acts to prevent the entire mass of the form from immediately disintegrating. The hydrated outer layer thereafter slowly dissolves releasing medicament. There can also be a release of medicament by leaching or diffusing action at or near the surface of the mass. As the outer surface dissolves or degrades, new surface is exposed to gastric fluid and the hydration region progressively moves through the volume of the form to the interior. This happens in a gradual process so as to maintain the integrity of the barrier. This process is continuously repeated until all or substantially all of the medicament is released. Thereafter, any remaining matrix which is still buoyant in gastric fluid slowly dissolves and is eliminated.

It has been found that the release pattern attained with the sustained release formulation of preferred embodiments herein has advantages over other sustained release mechanisms known in the art, particularly wherein the medicament contained therein is principally absorbed and/or exerts its therapeutic activity in the stomach or duodenum. Sustained or extended release forms such as capsules, granules, pellets and tablets prepared in accordance with certain preferred embodiments unexpectedly produce zero-order release after initial quick release of active ingredients. In addition, preferred sustained release formulations provide an excellent means for administering antacid, anti-viral, and anti-microbial substances, as well as other preferably acid-stable medicaments, over a prolonged period of time.

Polymers suitable for the optional polymer component in certain embodiments of the sustained release formulations disclosed herein optionally include one or more polymers including hydrophilic gums, modified celluloses and proteinaceous materials, which polymers may be anionic or nonionic and either natural or partially or totally synthetic. Examples of such materials include acacia, gum tragacanth, locust bean gum, tara, karaya gum, agar, guar gum, konjac, gum ghatti, pectin, carrageenan, xanthan gum, soluble and insoluble alginates, methylcellulose, hydroxypropylmethylcellulose, hydroxypropylcellulose, hydroxyethylcellulose, sodiumcarboxymethylcellulose, carboxypolymethylene, gelatin, zein, and the like. One preferred polymer for use in accordance with preferred embodiments is hydroxypropylmethylcellulose (HPMC). These materials may be used dry or as suspensions. or hydrocolloids. Optional polymers preferably hydrate in acidic medium having a pH of approximately 1, such as gastric fluid. The optional polymer can be chosen to affect the rate of medicament release from the form following ingestion. High viscosity materials, such as methylcellulose 60 HG 4000 cps, hydrate more slowly and maintain a soft mass for a longer time than lower viscosity materials, such as methylcellulose 60 HG 10 cps.

Swellable inorganic materials and colloids such as bentonite, colloidal magnesium aluminum silicate (Veegum) and the like, may also be used.

In addition to the milk proteins, and with or without other polymers, the extended release compositions according to preferred embodiments optionally comprise one or more hydrophobic or fatty materials. Preferred hydrophobic or fatty materials have a specific gravity of less than one and/or are substantially pharmaceutically inert. Such material can be added to the formulation to decrease the hydrophilic properties and to increase buoyancy. Examples of preferred fatty materials include, but are not limited to, waxes, such as carnauba wax and beeswax; fatty acids; long chain fatty alcohols such as, cetyl alcohol, myristyl alcohol, and stearyl alcohol; glycerides such as glyceryl esters of fatty acids or hydrogenated aliphatic acids such as, glyceryl monostearate, glyceryl distearate, glyceryl esters of hydrogenated castor oil and the like; and oils such as mineral oil or vegetable oil.

The term “low molecular weight concentrated milk proteins” is used to describe a material comprising a mixture of milk proteins wherein the very high molecular weight components of milk, such as immunoglobulins, and the non-protein components, such as salt and lactose, have been substantially eliminated from the material and preferably comprise less than about 20%, preferably less than about 10% or about 5% of the material. In a preferred embodiment, substantially all of the remaining proteins have molecular weights of less than about 100,000 daltons, and/or about 90%, preferably about 95% of the lactose is removed. Such material may be obtained from milk or some fraction or portion of milk (e.g. milk proteins, pasteurized skimmed milk, etc.) using one or more separation methods including those known in the art, such as filtration or ultrafiltration through a membrane or molecular sieve that retards or excludes molecules having a size or molecular weight above a particular threshold, (e.g. 100,000 daltons), centrifugation or other suitable method. In one embodiment, concentration of the milk proteins is 10 to 100 fold higher, including about 30 fold higher, than in whole milk.

The sustained release formulations may also include further additives, including, but not limited to, excipients, diluents, preservatives, stabilizers, lubricants, fillers, colorants, flavors, and the like. The choice of which such materials to use, if any, and the amounts to be utilized are considered to be within the abilities of one skilled in the art, in view of the disclosure herein. However, additives which might adversely affect the buoyancy of the extended release formulation should either not be used or only be used in quantities insufficient to cause a substantial negative effect upon the buoyancy or other characteristics of the formulation.

The amount of the total amount of the one or more active drugs or medicaments in the sustained release formulation, can vary over a wide range, preferably from about 0.01% by weight to about 80% by weight, including about 2% by weight to about 50% by weight of the composition as a whole, including about 10%, 15%, 20%, 30%, 40%, 50%, 60%, 70% and 80% by weight and ranges encompassing and bordered by such amounts, and may depend upon several factors, including the desired rate of release of the drug, the chemical or physical properties of the drug such as hydrophilic or hydrophobic properties, the amount of the drug required to give full therapeutic dosage, the bulk density thereof, the stability thereof and the like. These properties are known or are easily ascertainable by a person skilled in the art, such that formulation adjustments required to incorporate a given therapeutically active substance into a sustained release tablet herein are considered to be within the purview of one skilled in the art in view of the disclosure herein.

The amount of the low molecular weight concentrated milk proteins component present in preferred sustained release formulations may also vary within a wide range, preferably from about 2% by weight to about 99% or nearly 100% by weight, including about 20% by weight to about 80% by weight, and about 40% by weight to about 80% by weight, including about 10%, 20%, 30%, 40%, 50%, 60%, 70%, 80% and 90% by weight and ranges encompassing and bordered by such amounts. The stated percentages are the percentages in the composition as a whole. The amount of low molecular weight concentrated milk proteins will vary depending upon several factors, including the amounts and properties of the active ingredient(s), and upon how much, if any, optional polymer, optional fatty material, and/or additives are present.

When the optional one or more polymers is present in the sustained release formulation, the total amount present may vary within a wide range, preferably from about 0.1% by weight to about 90% by weight, including about 2% to about 80% by weight, about 20% to about 80% by weight, and about 2% by weight to about 20% by weight of the total composition, including about 5%, 10%, 15%, 20%, 30%, 40%, 50%, 60%, 70% and 80% by weight and ranges encompassing and bordered by such amounts. These ranges refer, of course, to when such polymer component is present; otherwise the amount is 0%. The amount of the polymer component present may depend, at least in part, upon the amount and identity of each of the other components present, and the identity and properties of the particular polymer(s). When one or more fatty materials are present in the sustained release formulation, such fatty material preferably comprises up to about 40% by weight of the total formulation, including about 0.1% by weight to about 20% by weight, including about 0.1%, 0.5%, 1%, 5%, 10%, 20%, 30%, and 40% by weight and ranges encompassing and bordered by such amounts. The amount of fatty material utilized may be governed, at least in part, by the amounts and physical characteristics of the active ingredient and any optional polymer with the object being to achieve a hydrodynamically balanced formulation, preferably a formulation which acquires a bulk density (specific gravity) of less than one in gastric fluids.

The amount of edible, inert pharmaceutical additives which may be present in the sustained release formulations will also vary in accordance with the amounts and physical properties of the other ingredients. Materials having a bulk density of less than one, e.g., ethylcellulose, are preferred. It is possible to utilize the selection of additive materials to modify the rate of release of the formulation. For example, soluble excipients, such as sugars will generally increase the rate of drug release, and insoluble excipients, such as dicalcium phosphate and the like, will generally decrease the rate of drug release, largely due to effects on the solubility of the formulation. Other preferred additives include silicon dioxide and magnesium stearate. When present, the total amount of additives present can be up to 80% by weight of the final formulation. Generally, such additives are present in from about 0.1% by weight to about 60% by weight of the formulation, including about 0.1% to about 2%, about 0.5% to 5%, and 10% by weight to about 30% by weight, including about 0.1%, 0.5%, 1%, 5%, 10%, 20%, 30%, 40%, 50%, 60%, 70% and 80% by weight and ranges encompassing and bordered by such amounts. The inclusion of, choice of, and quantity of all optional materials, including polymers, fatty or hydrophobic materials, and additives, in the sustained release formulations is within the abilities of one skilled in the art in view of the disclosure herein.

The sustained release dosage forms may be prepared from the formulations by any suitable technique, including, but not limited to, those well known in the art. For example, tablets may be prepared by tabletting techniques. Tabletting may use dry granulation, or, where the physical properties of the ingredients will permit, tablets may be prepared by wet granulation of the mixture of the ingredients. Tablets preferably include lubricants and may also contain other additives and excipients as discussed hereinabove.

It is to be noted that low molecular weight concentrated milk proteins and many of the optional polymers which may be utilized in the practice of the invention are also useful in pharmaceutical compounding as tablet binders and, as such, can be incorporated into the tablet formulation in the form of a solution or dispersion in a suitable solvent. In preferred embodiments, however, the polymer ingredient is preferably incorporated into the formulation in “dry” form, and excluding it from wet granulation techniques, where they are utilized. However, a small percentage of the polymer ingredient may be utilized as a tablet binder. Wherein a polymer such as described herein is used as a tablet binder and is combined into the formulation in the presence of a solvent, such polymer does not generally function to facilitate the buoyancy of the tablets prepared therefrom.

When making tablets and other compressed forms, care should be taken in the pressing of the form, as the degree of compression and density of the resulting form will affect the rate of hydration and therefore the rate of release of the drug. For example, if the form is overly compressed, it will resist hydration in the stomach and may not achieve a bulk density sufficiently low to be buoyant in the stomach. It should be noted, however, that forms which initially have a density greater than one can be buoyant in gastric fluids due to a combination of an increase in the bulk volume of the form when it contacts gastric fluids due to the hydration and swelling of the milk protein and other polymer particles, especially those in the vicinity of the surface of the form. It will be appreciated that the maximum hardness to which a form having an initial density greater than one can be compressed will vary with factors such as the initial density of the formulation, the size of the form, and the composition of the formulation. The preferred hardness for a form will lie between the maximum at which a buoyant form can be produced in accordance with the teachings herein and a minimum required for the form to be physically stable, such as by substantially resisting flaking, crumbling and breaking during normal storage, shipment, and use. This range of hardness can be easily determined by known pharmaceutical hardness measurements combined with testing of the buoyancy of samples of tablets different hardness in gastric fluid or its equivalent.

The medicament or combination of medicaments which are amenable to sustained release therapy utilizing the novel formulations of the present invention include any of those suitable for oral administration where sustained release therapy is medically advisable. It is to be understood that the disclosure herein is not to be construed as being limited to any particular medicament or class of medicaments. Further, the sustained release tablet is not restricted to medicaments which are principally absorbed from the stomach since it has been found that it is equally efficacious with medicaments which are absorbed from the intestines, e.g., chlorpheniramine maleate. The sustained release dosage formulation has more limited usefulness with medicaments which are acid sensitive. Among the various medicaments and classes of medicaments which are advantageously administered via a sustained release dosage form are, for example, antimicrobials, antibacterials, antibiotics, e.g., penicillins, cephalosporins and tetracyclines, including ciprofloxacin, cyclosporin, cefoxitin, and minocycline; catecholamines, e.g., epinephrine and the amphetamines; antiamebics; antiasthmatics; anticholinergics; anorexics and anti-obesity drugs such as orlistat; antidepressants; benzodiazepines, e.g., chlordiazepoxide, diazepam, oxazepam, and bromazepam; antitussive; narcotic analgesics, e.g. codeine, oxycodone, oxymorphone, methadone; non-narcotic analgesics, e.g., aspirin, NSAIDs including ibuprofen, naproxen; COX-2 inhibitors, e.g. celecoxib, rofecoxib; antidiabetics such as metformin, anti-hyperglycemics, anti-hypoglycemics; anti-Parkinsonians such as selegiline, antivirals, e.g. acyclovir, ganciclovir; sedatives, e.g., the barbiturates; vasodilators such as nimodipine; diuretics; anticonvulsants; antiemetics, e.g. compazine, muscle relaxants such as baclofen; hypotensives and antihypertensives such as captopril and methyldopa; antifungals; anticholesterolemics; antacids, protein pump inhibitors, and H2 antagonists e.g., omeprazole, lansoprazole, esomeprazole, ranitidine, cimetidine; antihistamines such as fexofenadine; and vitamins. Other suitable medicaments include allopurinol, furosemide, 5-aminosalicylate, moexipril, and bupropion. It is reported in the literature that the irritation of the stomach caused by aspirin is the result of contact of this very acidic substance with the stomach walls. Therefore, it will be appreciated that the formulations according to certain preferred embodiments herein are particularly advantageous for the administration of aspirin since they remain buoyant in gastric fluid.

The sustained release formulations are also particularly amenable to the administration of medicaments which are only absorbed through the stomach or upper portion of the intestines, e.g., ferrous salts such as ferrous fumarate, or which exert a therapeutic effect in the stomach, for example, antacids such as the oxides, hydroxides and carbonates of magnesium, aluminum hydroxide, magnesium trisilicate and the like. Wherein such substances generate carbon dioxide, some bubbles will become entrapped by the hydrated outer layer thus enhancing the buoyancy of the tablet. Similarly, amounts of carbon dioxide generating bases can also be utilized in non-antacid formulations to enhance buoyancy.

It is further within the scope of the disclosure herein to administer the formulations hydrodynamically balanced as one layer of a two layer form. The remaining layer contains medicament in a formulation generally free of sustained release properties and/or ingredients. This unique form, includes an outer layer which upon ingestion, provides an immediate release of medicament and an inner buoyant layer which continues to release medicament over a period of time while being retained in the stomach. Such unique two-layered tablets are particularly advantageous for the administration of antacid substances alone or in combination with other drugs, including certain analgesics, which cause stomach irritation.

The following examples further illustrate preferred embodiments of the invention.

### EXAMPLE 1

Caffeine tablets were prepared from the following formulation:

| Ingredient | mg/tablet |
|---|---|
| Caffeine | 100.0 |
| Hydroxypropyl Methylcellulose K100 | 65.0 |
| low molecular weight concentrated milk proteins | 500.0 |
| Colloidal Silicon Dioxide | 6.0 |
| Magnesium Stearate | 6.0 |
| Total | 677.0 |

The caffeine was passed through a Fitzpatrick Comminuting Machine using a #00 screen, hammers forward. The milled material was combined with hydroxypropyl Methylcellulose and low molecular weight concentrated milk proteins mixture and thoroughly mixed. The colloidal silicon dioxide and magnesium stearate were then added and the total mixture homogeneously blended and compressed into tablets using standard caplet shape punches. The tablets were compressed to a hardness of 10-14 Kp and it was determined that the hardness should preferably not exceed 16 Kp. It was found that tablets at hardness less than 14 Kp would float instantly on artificial gastric fluid, whereas tablets at 16 Kp would sink temporarily before rising to the surface. Tablets at more than 25 Kp were not buoyant.

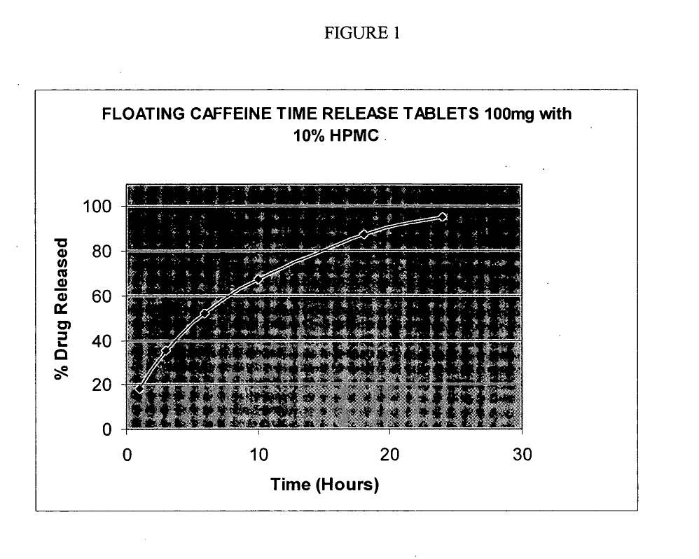

In vitro release tests were carried out by the modified N.F. method in gastric fluid only at 50 r.p.m. The results are set forth in the following table and illustrated in FIG. 1.

TABLE I
PERCENT ACTIVE INGREDIENT RELEASED

| Time (Hours) | Average | Range |
|---|---|---|
| 1 | 18.3 | 17.9–18.7 |
| 3 | 35.5 | 34.9–36.1 |
| 6 | 52.1 | 51.5–53.0 |
| 10 | 67.3 | 66.5–68.0 |
| 18 | 87.4 | 86.5–88.3 |
| 24 | 95.4 | 94.4–96.1 |

### EXAMPLE 2

Caffeine tablets containing 0% hydroxypropyl methylcellulose were prepared from the following formulation:

| Ingredient | mg/tablet |
|---|---|
| Caffeine | 100.0 |
| low molecular weight concentrated milk proteins | 500.0 |
| Colloidal Silicon Dioxide | 5.0 |
| Magnesium Stearate | 5.0 |
| Total | 610.0 |

The caffeine was passed through a Fitzpatrick Comminuting Machine using a #00 screen, hammers forward. The milled material was combined with low molecular weight concentrated milk proteins and thoroughly mixed. The colloidal silicon dioxide and magnesium stearate were then added and the total mixture homogeneously blended and compressed into tablets using standard caplet shape punches. The tablets were compressed to a hardness of 10-14 Kp and it was determined that the hardness preferably does not exceed 16 Kp. It was found that tablets at hardness less than 14 Kp would float instantly on artificial gastric fluid, whereas tablets at 16 Kp would sink temporarily before rising to the surface. Tablets at more than 25 Kp were not buoyant.

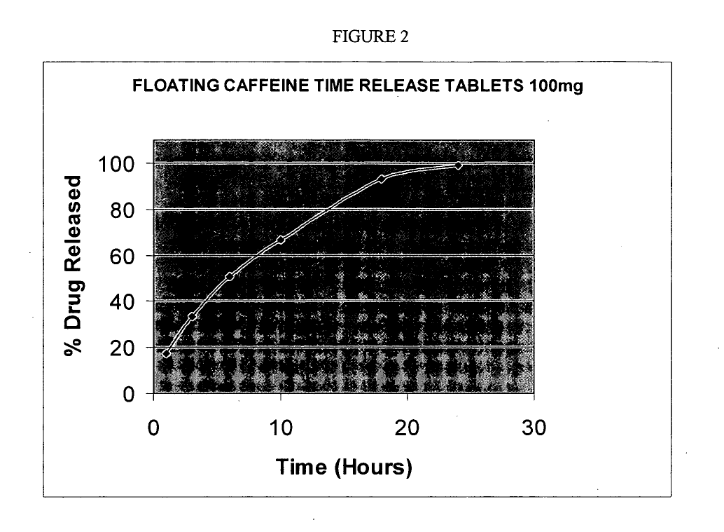

In vitro release tests were carried out by the modified N.F. method in gastric fluid only at 50 r.p.m. The results are set forth in the following table and illustrated in FIG. 2.

TABLE II
PERCENT ACTIVE INGREDIENT RELEASED

| Time (Hours) | Average | Range |
|---|---|---|
| 1 | 17.2 | 16.7–17.7 |
| 3 | 33.5 | 33.1–34.3 |
| 6 | 50.6 | 50.0–51.3 |
| 10 | 66.8 | 66.1–67.5 |
| 18 | 92.9 | 90.3–93.5 |
| 24 | 98.9 | 98.2–99.9 |

### EXAMPLE 3

Guaifenesin tablets containing 0% hydroxypropyl methylcellulose were prepared from the following formulation:

| Ingredient | mg/tablet |
|---|---|
| Guaifenesin | 600.0 |
| low molecular weight concentrated milk proteins | 500.0 |
| Colloidal Silicon Dioxide | 12.0 |
| Magnesium Stearate | 15.0 |
| Total | 1127.0 |

The guaifenesin was passed through a Fitzpatrick Comminuting Machine using a #1 screen, knives forward. The milled material was combined with low molecular weight concentrated milk proteins and thoroughly mixed. The colloidal silicon dioxide and magnesium stearate were then added and the total mixture homogeneously blended and compressed into tablets using standard caplet shape punches. The tablets were compressed to a hardness of 6-10 Kp and it was determined that the hardness preferably does not exceed 12 Kp. It was found that tablets at hardness less than 10 Kp would float instantly on artificial gastric fluid, whereas tablets at 12 Kp would sink temporarily before rising to the surface. Tablets at more than 20 Kp were not buoyant.

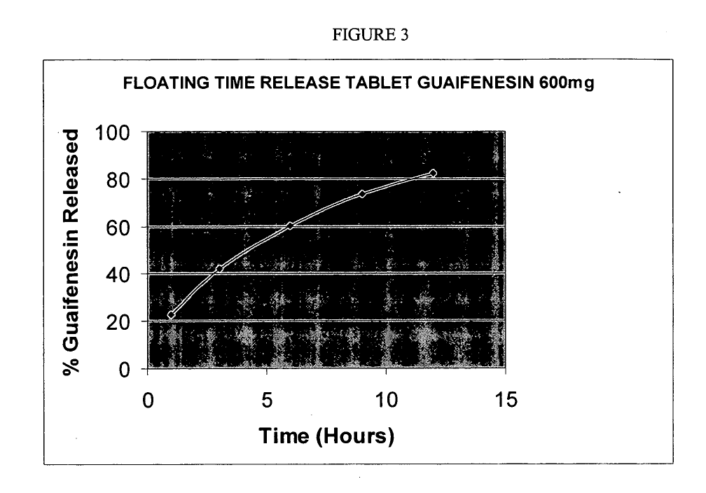

In vitro release tests were carried out by the modified N.F. method in gastric fluid only at 75 r.p.m. The results are set forth in the following table and illustrated in FIG. 3.

TABLE III
PERCENT ACTIVE INGREDIENT RELEASED

| Time (Hours) | Average | Range |
|---|---|---|
| 1 | 22.9 | 21.5–24.1 |
| 3 | 42.0 | 39.9–43.4 |
| 6 | 60.4 | 57.5–62.6 |
| 9 | 73.6 | 70.1–76.3 |
| 12 | 82.4 | 79.1–84.8 |

### EXAMPLE 4

Anti-cough and cold tablets containing pseudoephedrine HCl, methscopolamine nitrate, chlorpheniramine maleate and 0% hydroxypropyl methylcellulose were prepared from the following formulation:

| Ingredient | mg/tablet |
|---|---|
| Pseudoephedrine HCl | 91.837 |
| Methscopolamine Nitrate | 2.021 |
| Chlorpheniramine Maleate | 8.00 |
| low molecular weight concentrated milk proteins | 500.0 |
| Colloidal Silicon Dioxide | 5.0 |
| Magnesium Stearate | 5.0 |
| Total | 611.858 |

The active ingredients—pseudoephedrine HCl (PSE), methscopolamine nitrate (METH), chlorpheniramine maleate (CPM) were passed through a Fitzpatrick Comminuting Machine using a #000 screen, hammers forward. The milled material was combined with low molecular weight concentrated milk proteins and thoroughly mixed. The colloidal silicon dioxide and magnesium stearate were then added and the total mixture homogeneously blended and compressed into tablets using standard caplet shape punches. The tablets were compressed to a hardness of 10-14 Kp and it was determined that the hardness preferably does not exceed 16 Kp. It was found that tablets at hardness less than 14 Kp would float instantly on artificial gastric fluid, whereas tablets at 16 Kp would sink temporarily before rising to the surface. Tablets at more than 25 Kp were not buoyant.

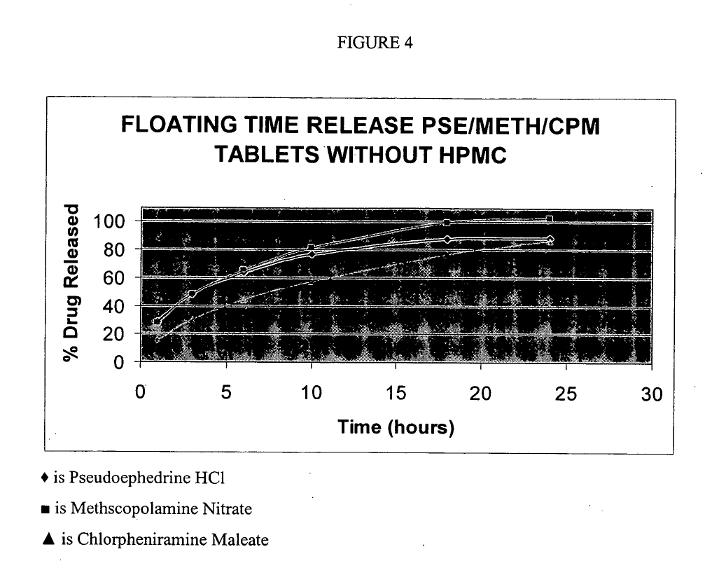

In vitro release tests were carried out by the modified N.F. method in gastric fluid only at 50 r.p.m. The results are set forth in the following table and illustrated in FIG. 4, where the PSE average is shown by the diamond shape, METH the square, and SPM the triangle shape.

TABLE IV-A
PERCENT ACTIVE INGREDIENT RELEASED

| Time (Hours) | PSE Average | PSE Range |
|---|---|---|
| 1 | 28.6 | 28.1–29.4 |
| 3 | 47.9 | 46.6–48.7 |
| 6 | 63.6 | 62.0–64.3 |
| 10 | 76.6 | 75.6–77.9 |
| 18 | 87.6 | 86.6–88.4 |
| 24 | 88.3 | 87.4–89.0 |

TABLE IV-B
PERCENT ACTIVE INGREDIENT RELEASED

| Time (Hours) | METH Average | METH Range |
|---|---|---|
| 1 | 28.3 | 25.4–32.2 |
| 3 | 48.3 | 45.6–52.8 |
| 6 | 65.4 | 58.7–71.0 |
| 10 | 81.1 | 73.8–88.5 |
| 18 | 99.6 | 91.2–104.3 |
| 24 | 102.8 | 98.4–104.7 |

TABLE IV-C
PERCENT ACTIVE INGREDIENT RELEASED

| Time (Hours) | CPM Average | CPM Range |
|---|---|---|
| 1 | 15.7 | 15.6–16.0 |
| 3 | 30.0 | 28.9–30.7 |
| 6 | 44.0 | 42.5–44.9 |
| 10 | 57.5 | 56.3–58.7 |
| 18 | 76.9 | 74.8–78.6 |
| 24 | 86.6 | 84.0–89.0 |

### EXAMPLE 5

Anti-cough and cold tablets containing pseudoephedrine HCl, methscopolamine nitrate, chlorpheniramine maleate and 10% hydroxypropyl methylcellulose were prepared from the following formulation:

| Ingredient | mg/tablet |
|---|---|
| pseudoephedrine HCl | 91.837 |
| Methscopolamine Nitrate | 2.021 |
| Chlorpheniramine Maleate | 8.00 |
| low molecular weight concentrated milk proteins | 500.0 |
| Hydroxypropyl Methylcellulose K100 | 68.0 |
| Colloidal Silicon Dioxide | 5.0 |
| Magnesium Stearate | 5.0 |
| Total | 679.858 |

The active ingredients—pseudoephedrine HCl (PSE), methscopolamine nitrate (METH), chlorpheniramine maleate (CPM) were passed through a Fitzpatrick Comminuting Machine using a #000 screen, hammers forward. The milled material was combined with low molecular weight concentrated milk proteins and thoroughly mixed. The colloidal silicon dioxide and magnesium stearate were then added and the total mixture homogeneously blended and compressed into tablets using standard caplet shape punches. The tablets were compressed to a hardness of 10-14 Kp and it was determined that the hardness preferably does not exceed 16 Kp. It was found that tablets at hardness less than 14 Kp would float instantly on artificial gastric fluid, whereas tablets at 16 Kp would sink temporarily before rising to the surface. Tablets at more than 25 Kp were not buoyant.

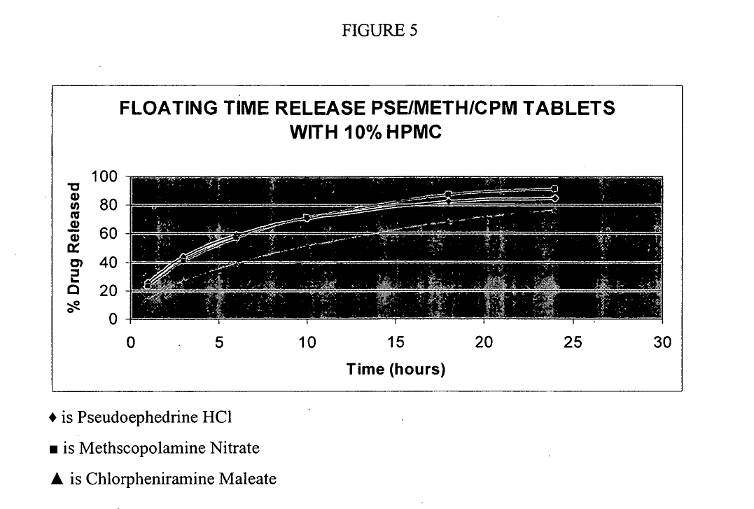

In vitro release tests were carried out by the modified N.F. method in gastric fluid only at 50 r.p.m. The results are set forth in the following table and illustrated in FIG. 5.

TABLE V-A
PERCENT ACTIVE INGREDIENT RELEASED

| Time (Hours) | PSE Average | PSE Range |
|---|---|---|
| 1 | 26.0 | 25.2–26.8 |
| 3 | 43.7 | 43.0–45.2 |
| 6 | 58.9 | 57.9–60.4 |
| 10 | 70.8 | 69.9–72.3 |
| 18 | 82.7 | 81.5–84.3 |
| 24 | 84.9 | 83.6–86.8 |

TABLE V-B
PERCENT ACTIVE INGREDIENT RELEASED

| Time (Hours) | METH Average | METH Range |
|---|---|---|
| 1 | 23.4 | 21.6–24.9 |
| 3 | 41.1 | 37.8–44.6 |
| 6 | 57.3 | 52.8–61.7 |
| 10 | 71.5 | 65.8–75.7 |
| 18 | 87.4 | 80.9–91.7 |
| 24 | 91.4 | 85.2–96.3 |

TABLE V-C
PERCENT ACTIVE INGREDIENT RELEASED

| Time (Hours) | CPM Average | CPM Range |
|---|---|---|
| 1 | 14.0 | 13.6–14.2 |
| 3 | 26.7 | 26.2–27.2 |
| 6 | 39.4 | 38.8–40.0 |
| 10 | 51.6 | 50.9–52.3 |
| 18 | 68.6 | 67.5–70.6 |
| 24 | 76.7 | 75.4–79.2 |

It will be understood that the specification and examples are illustrative but not limitative of the disclosed invention and that other embodiments within the spirit and scope of the invention will suggest themselves to those skilled in the art.

What is claimed is:

1. A pharmacologically active gastric buoyant composition having extended gastric residence time with sustained drug release, said composition comprising:
at least one pharmacologically active compound,
at least one pharmacologically acceptable additive, and
low molecular weight concentrated milk proteins;
wherein the composition is compressed to a hardness of less than about 25 kp.

2. The composition of claim 1 wherein the composition further comprises one or more polymers.

3. The composition of claim 2 wherein the weight ratio of low molecular weight concentrated milk proteins to polymer ranges from 20:80 to 100:0.

4. The composition of claim 2 wherein the one or more polymers are hydrophilic.

5. The composition of claim 2 wherein the one or more polymers have a number average molecular weight of between about 1,000 and 20,000,000 grams per mole.

6. The composition of claim 2 wherein the one or more polymers are selected from the group consisting of polyethylene oxide, hydroxypropyl cellulose, hydroxypropyl methyl cellulose, hydroxyethyl cellulose, sodium carboxy methylcellulose, calcium carboxymethyl cellulose, methyl cellulose, ethyl cellulose, polyacrylic acid, methacrylic acid, methyl methacrylate, acrylic and methacrylic acid esters, polyvinylpyrrolidone, maltodextrin, pre-gelatinized starch and polyvinyl alcohol.

7. The composition of claim 1, further comprising one or more fatty materials.

8. The composition of claim 1, wherein the active compound comprises at least one compound selected from the group consisting of ciprofloxacin, nimodipine, captopril, ranitidine, cyclosporin, baclofen, allopurinol, furosemide, cefoxitine, 5-aminosalicylate and moexipril.

9. The composition of claim 1, wherein the active compound is an antacid.

10. The composition of claim 1, wherein the active compound is selected from the group consisting of antitussive, antiviral, antimicrobial, antidiabetic, anti-hyperglycemic anti-hypoglycemic, antihypertension, antidepressant, anorexic, and antifungal active agents.

11. The composition of claim 10, wherein the active compound is selected from the group consisting of acyclovir, ganciclovir, cimetidine, ranitidine, captopril, methyldopa, selegiline, minocycline, metformin, bupropion, orlistat, fexofenadine and pharmaceutically acceptable salts thereof.

12. The composition of claim 1, wherein the active compound is selected from the group consisting of catecholamines, antiasthmatics, anticholinergics, benzodiazepines, narcotic analgesics, non-narcotic analgesics, COX-2 inhibitors, sedatives, anticonvulsants, antiemetics, muscle relaxants, anticholesterolemics, antacids, protein pump inhibitors, and H2 antagonists.

13. The composition of claim 1 wherein the dosage form is adapted to deliver in the stomach, as a single dose and over a prolonged time period, a therapeutically-effective amount of the active agent.

14. The composition of claim 13 wherein the prolonged time period is at least 4 hours.

15. The composition of claim 14 wherein the prolonged time period is between about 8 to 24 hours.

16. The composition of claim 14 wherein the prolonged time period is between about 12 to 48 hours.

17. The composition of claim 1 wherein the composition is compressed to a hardness of about 10-14 kp.

18. The composition of claim 1 wherein the molecular weight concentrated milk proteins have molecular weights of less than about 100,000 daltons.

19. A pharmacologically active gastric buoyant composition having extended gastric residence time with sustained drug release, said composition comprising:
at least one pharmacologically active compound,
at least one pharmacologically acceptable additive,
low molecular weight concentrated milk proteins, and
one or more polymers.

20. The composition of claim 19 wherein the composition is compressed to a hardness of less than about 25 kp.

21. The composition of claim 19 wherein the composition is compressed to a hardness of about 10-14 kp.

22. The composition of claim 19 wherein the low molecular weight concentrated milk proteins and polymer are present in the form of a substantially homogeneous mixture in which the weight ratio of low molecular weight concentrated milk proteins to polymer is from 20:80 to 100:0.

23. The composition of claim 19 wherein the low molecular weight concentrated milk proteins and polymer are present in the form of a homogeneous mixture.

24. The composition of claim 19 wherein the one or more polymers are swellable.

25. The composition of claim 19 wherein the one or more polymers are hydrophilic.

26. The composition of claim 19 wherein the one or more polymers are hydrophobic.

27. The composition of claim 19 wherein the one or more polymers have a number average molecular weight of between about 1,000 and 20,000,000 grams per mole.

28. The composition of claim 19 wherein the molecular weight concentrated milk proteins have molecular weights of less than about 100,000 daltons.

29. The composition of claim 19 wherein the weight percent of the low molecular weight concentrated milk proteins in the composition is about 20 to 99 weight percent.

30. The composition of claim 19 wherein the weight percent of the one or more polymer in the matrix is about 2 to 80 weight percent.

31. The composition of claim 1, further comprising one or more fatty materials.

32. The composition of claim 31 wherein the weight percent of the fatty material is about 0.1 to 40 weight percent.

33. The composition of claim 19 wherein the one or more polymers are selected from the group consisting of polyethylene oxide, hydroxypropyl cellulose, hydroxypropyl methyl cellulose, hydroxyethyl cellulose, sodium carboxy methylcellulose, calcium carboxymethyl cellulose, methyl cellulose, ethyl cellulose, polyacrylic acid, methacrylic acid, methyl methacrylate, acrylic and methacrylic acid esters, polyvinylpyrrolidone, maltodextrin, pre-gelatinized starch and polyvinyl alcohol.

34. A pharmacologically active gastric buoyant composition having extended gastric residence time with sustained drug release, said composition comprising:
at least one pharmacologically active compound,
one or more fatty materials,
low molecular weight concentrated milk proteins, and
one or more polymers.

35. The composition of claim 34 wherein the composition is compressed to a hardness of less than about 25 kp.

36. The composition of claim 34 wherein the composition is compressed to a hardness of about 10-14 kp.

37. The composition of claim 34 wherein the low molecular weight concentrated milk proteins and polymer are present in the form of a substantially homogeneous mixture in which the weight ratio of low molecular weight concentrated milk proteins to polymer ranges from 20:80 to 100:0 and/or the one or more polymers have a number average molecular weight of between about 1,000 and 20,000,000 grams per mole.
